(19) United States
(12) Patent Application Publication
(10) Pub. No.: US 2004/0047895 A1
(43) Pub. Date: Mar. 11, 2004

(54) Laver protein-containing composition and foods

(75) Inventors: Hiroshi Hagino, Tokyo (JP); Masanobu Saito, Chiba-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: SHIRAKO CO., LTD., Tokyo (JP)

(21) Appl. No.: 10/652,069

(22) Filed: Sep. 2, 2003

(30) Foreign Application Priority Data
Sep. 5, 2002 (JP) 2002-259922

(51) Int. Cl. A61K 35/80; A61K 47/00; C07K 14/405

(52) U.S. Cl. 424/439; 424/195.17; 530/395

(57) ABSTRACT

This invention provides a composition capable of efficiently exhibiting various kinds of physiological activities possessed potentially by seaweeds of the genus Porphyra. The laver protein-containing composition is obtained by adding water, a saline solution or an aqueous dilute alkali solution to seaweeds of the genus Porphyra or finely divided dry particles thereof, wet milling the materials to extract soluble components therefrom, and separating proteins form the extract. A composition containing different kinds of laver proteins may be obtained by separately conducting extraction with water, a saline solution or an aqueous dilute alkali solution, or a composition containing a mixture of laver proteins may be obtained by conducting such extraction procedures successively. The laver protein-containing composition thus obtained is used as a food helpful to health because it has a blood pressure-dropping action, a hepatic function-improving action, a lipid metabolism-improving action, a peripheral blood vessel-expanding action and a blood viscosity-reducing action.

### BACKGROUND OF THE INVENTION

1. Field of the Invention

The present invention relates to a laver protein-containing composition obtained from seaweeds of the genus Porphyra and to foods using the same.

2. Description of the Related Art

Seaweeds of the genus Porhyra propagate worldwide in a wide area, and in Japan, cultured Porohyra yezoensis is consumed as edible seaweed in a large amount. As eating habits are changed in recent years, consumption of edible seaweeds shows a downward tendency, but development thereof for other uses is advancing.

It is said that seaweeds of the genus Porphyra abundantly contain nutriments such as proteins, dietary fibers, vitamins, minerals etc., and are foods helpful to health. Further, it is noted that the seaweeds of the genus Porphyra have physiological activities such as a blood pressure-dropping action, a hepatic function-improving action, a lipid metabolism-improving action, a peripheral blood vessel-expanding action and a blood viscosity-reducing action.

It was revealed that these physiological activities possessed by the seaweeds of the genus Porphyra are exerted mainly by peptides produced from proteins in the seaweeds of the genus Porphyra through decomposition with an enzyme pepsin in gastric juice. However, the digestibility of the seaweeds of the genus Porphyra is not necessarily high because the seaweeds have strong cell walls. Further, the rate of decomposition of proteins from the seaweeds of the genus Porphyra with pepsin is also low. Accordingly, when the seaweeds of the genus Porphyra or a dry product thereof are ingested as such, the seaweeds or a dry product thereof should be eaten in a very large amount in order to achieve these physiological activities, and actually such ingestion is poor in efficiency.

### SUMMARY OF THE INVENTION

The present invention was made in view of the circumstances described above, and the object of the present invention is to attempt at utilizing seaweeds of the genus Porphyra effectively by preparing proteins thereof in order to efficiently exhibit physiological activities possessed potentially by the seaweeds of the genus Porphyra.

The present invention was made to achieve the object described above, and relates to a laver protein-containing composition obtained by adding water, a saline solution or an aqueous dilute alkali solution to seaweeds of the genus Porphyra or finely divided dry particles thereof, wet milling the materials to extract soluble components therefrom, and separating proteins from the extract. Further, the present invention relates to health foods comprising the laver protein-containing composition as a major component and having a blood pressure-dropping action, a hepatic function-improving action, a lipid metabolism-improving action, a peripheral blood vessel-expanding action and a blood viscosity-reducing action.

As described above, the seaweeds of the genus Porphyra have various physiological actions helpful to health, but the seaweeds themselves are poor in digestibility, and proteins contained therein are poor in degradability in the living body, and thus these are actually not effectively ingested. From the seaweeds of the genus Porphyra, proteins are separated efficiently in the present invention, so that the useful physiological activities of the proteins can be utilized efficiently thus contributing to maintaining and promoting health.

### BRIEF DESCRIPTION OF THE DRAWING

FIG. 1 shows an example of a process of producing the laver protein-containing composition of the present invention.

### DETAILED DESCRIPTION OF THE INVENTION

In the present invention, the laver protein-containing composition is obtained by wet milling the starting seaweeds in water, a saline solution or an aqueous dilute alkali solution to extract proteins at a high degree of extraction therefrom, and then separating laver proteins from the extract by a usual protein separation method. The protein separation method used in the present invention includes, for example, a protein precipitation method using an organic solvent such as ethanol, polyethylene glycol etc. or ammonium sulfate, an ion-exchanger adsorption method, an isoelectric precipitation method, a membrane separation method etc., and these methods can be used alone or in combination thereof.

The laver protein-containing composition has physiological activities such as a blood pressure-dropping action, a hepatic function-improving action, a lipid metabolism-improving action, a peripheral blood vessel-expanding action and a blood viscosity-reducing action, and is thus useful as food of very high nutritive value for maintaining health.

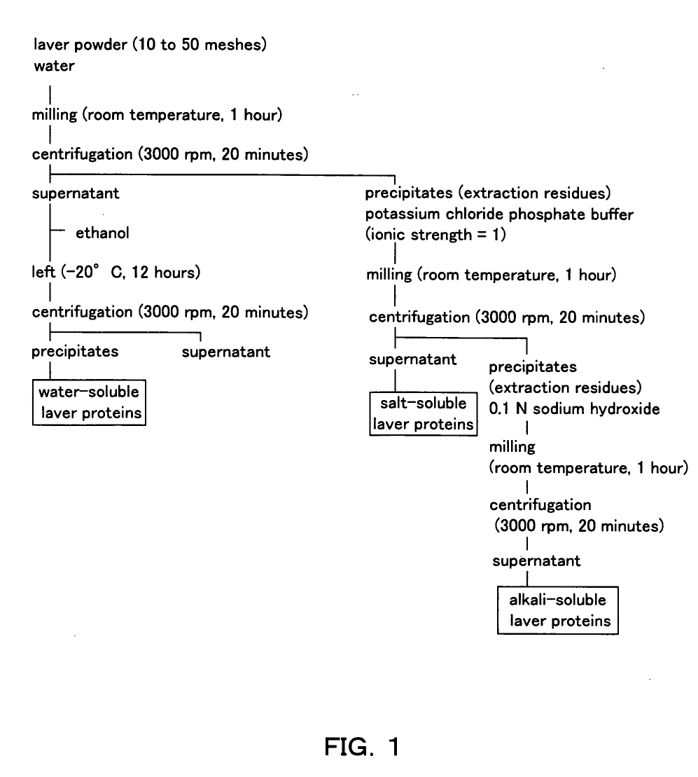

The process of producing the laver protein-containing composition of the present invention is described by reference to FIG. 1.

For example, 10 ml water is added to 2 g dry laver powder (10 to 50 meshes) which is then mixed and milled with an automatic mill at room temperature for 1 hour. Then, the milled material is separated into laver extraction residues and a supernatant by centrifugation with a centrifuge (3000 r.p.m.) for 20 minutes. Ethanol is added to the resulting supernatant, then left at −20° C. for 12 hours to precipitate proteins and centrifuged in a centrifuge (3000 r.p.m.) for 20 minutes, to give water-soluble laver proteins as precipitates.

On one hand, a potassium chloride-phosphoric acid buffer solution (ionic strength=1, pH 7.5) is added as a saline solution to the above laver extraction residues which are then milled and extracted in the same manner as above, whereby salt-soluble laver proteins can be obtained. The extraction residues from which the salt-soluble laver proteins were extracted are milled and extracted in the same manner with 0.1 N sodium hydroxide as a dilute alkali solution, whereby alkali-soluble laver proteins can be obtained.

These laver proteins may be used separately as water-soluble proteins, salt-soluble proteins and alkali-soluble proteins or may be used together. The means of separating and purifying the laver proteins includes the method using ethanol as described above and usual protein separation methods such as a protein precipitation method using an organic solvent such as polyethylene glycol etc. or ammonium sulfate, an ion-exchanger adsorption method, an isoelectric precipitation method, a membrane separation method etc., and these can be used alone or in combination thereof.

### EXAMPLES

### Example 1

Preparation of Water-Soluble Laver Proteins from Dry Laver

Dry laver prepared by drying cultured laver Porphyra yezoensis was finely divided into 35-mesh-passing powder with a high-speed pulverizer. 20 g of the finely divided powder was muddled in 400 ml distilled water, milled with a wet mill, then centrifuged for 20 minutes in a centrifuge (3000 r.p.m.) to give 100 ml laver protein-containing solution. 800 ml ethanol was added to this solution and left at −20° C. for 12 hours to precipitate proteins which were then centrifuged in a centrifuge (3000 r.p.m.) for 20 minutes to give precipitates. The precipitates were air-dried to give 5 g of water-soluble laver proteins.

### Example 2

Preparation of Water-Soluble Laver Proteins from Living Laver Seaweeds

200 g finely divided particles obtained by finely dividing living laver seaweeds with a homogenizer were suspended in 400 ml of 0.1 N sodium hydroxide, milled in a wet mill and centrifuged in a centrifuge (10000 r.p.m.) for 20 minutes, to give 250 ml alkali-soluble laver protein-containing solution. Then, the extraction residues were milled in 400 ml distilled water and centrifuged in a centrifuge (3000 r.p.m.) for 20 minutes, to give 25 ml water-soluble laver protein-containing solution.

The alkali-soluble laver protein-containing solution and the water-soluble laver protein-containing solution were mixed followed by removing low-molecular substances such as salts by a dialysis membrane to give a protein fraction solution, and the solution was lyophilized to give 11 g water- and alkali-soluble laver proteins.

### Test Example 1

Digestion Test

0.5 g each of the laver proteins obtained in Examples 1 and 2 were dissolved in 10 ml of 1/50 N hydrochloric acid containing 0.1% pepsin, and then left at 37° C. for 3 hours. Then, each solution was adjusted to pH 7.7 with 10% sodium carbonate, then 0.01 g pancreatin was added thereto, and the mixture was left at 37° C. for 20 hours. After non-decomposed substances were removed from each test solution through an ultrafiltration membrane having a cutoff molecular weight of 3000, the test solution was lyophilized to give a digested product of the laver proteins.

As the control for comparison, finely divided powder of dry laver was subjected to digestion with 1/50 N hydrochloric acid containing pepsin and then with pancreatin, removal of non-decomposed substances and lyophilization in the same manner as above to give a digested product.

Each digested product obtained from the laver proteins in Examples 1 and 2 and the control product were measured for their nitrogen content, and the nitrogen content (%) relative to that before digestion was determined. Each digested product was measured for its inhibitory activity on angiotensin I converting enzyme (ACE). The results are shown in Table 1.

TABLE 1

| | Nitrogen in the | |
|---|---|---|
| | artificially | Inhibitory |
| | digested product/ | activity on |
| | nitrogen in the | angiotensin I |
| | test sample | converting enzyme |
| | (%) | IC50 (mg/ml) |
| Water-soluble laver proteins | 98.2 | 0.87 |
| in Example 1 | | |
| Water- and alkali-soluble | 92.4 | 0.61 |
| laver proteins in Example 2 | | |
| Finely divided dry powder of | 52.6 | 3.01 |
| laver | | |

As can be seen from Table 1, the laver proteins in Examples 1 and 2 showed (nitrogen in the artificially digested product)/(nitrogen in the test sample) ratios of 98.2% and 92.4% respectively, indicating that a majority of nitrogen in the test sample is transferred to the digested product and a majority of the proteins are digested, while the finely divided dry powder of laver as the control showed a nitrogen ratio of 52.6%, indicating that nearly half of nitrogen remains without being digested.

Further, the ACE inhibitory activity indicative of blood pressure-dropping action was significantly stronger in the digested product of the laver proteins than in the digested product of the finely divided dry laver.

### Test Example 2

Blood Pressure-Dropping Action on Rats

Fifteen-week-old male rats with spontaneous hypertension were used as experimental animals. These rats were preliminarily raised for 2 weeks, and those having a systolic blood pressure of 190 mmHg or more were selected, and 6 animals were used as one group in the test.

The laver proteins obtained in Example 1 and the finely divided powder of laver (control) were orally administered in doses of 10 mg/kg and 30 mg/kg once into rat groups respectively. Using an ambulatory tail artery blood pressure monitoring device (MK-1030, manufactured by Muromachi Kikai Co., Ltd.), systolic blood pressure was measured 5 times by a tail-cuff method before administration, 1 hour, 2 hours, 3 hours, 4 hours, 6 hours and 8 hours after administration respectively, and the mean of 3 measurements excluding the maximum and minimum measurements was used as measurements at each point in time. The results are shown in Table 2.

TABLE 2

| | | Systolic blood pressure (mmHg) | | | | | |
|---|---|---|---|---|---|---|---|
| | | | 1 hour | 2 hours | 4 hours | 6 hours | 8 hours |
| | | before | after | after | after | after | after |
| | Dose | adminis- | adminis- | adiminis- | adminis- | adminis- | adminis- |
| | (mg/kg) | tration | tration | tration | tration | tration | tration |
| Water-soluble | 10 | 190 ± 5.6 | 191 ± 6.8 | 178 ± 4.2 | 165 ± 4.5 | 171 ± 3.2 | 185 ± 6.2 |
| laver proteins | 30 | 192 ± 7.1 | 182 ± 4.1 | 170 ± 3.3 | 161 ± 2.8 | 165 ± 1.8 | 174 ± 5.8 |
| in Example 1 | | | | | | | |
| Finely divided | 10 | 191 ± 5.3 | 192 ± 8.1 | 190 ± 7.19 | 185 ± 8.6 | 190 ± 4.7 | 190 ± 8 |
| dry powder of | 30 | 189 ± 4.6 | 187 ± 6.7 | 188 ± 4.9 | 180 ± 4.7 | 188 ± 7.7 | 191 ± 5.2 |
| laver | | | | | | | |

(Mean ± standard error)

As shown in Table 2, the group administered 10 mg/kg laver proteins obtained in Example 1 was confirmed to exhibit a significant reduction in blood pressure from 2 to 6 hours after administration. The group administered 30 mg/kg laver proteins was also confirmed to exhibit a significant reduction in blood pressure from 1 hour after administration. On the other hand, the group administered the finely divided dry powder of laver in a dose of 30 mg/kg was observed to exhibit a slight reduction in blood pressure.

### Test Example 3

Plasma Lipid Component Level-Lowering Action on Mice

Four-week-old male ICR strain mice were used as experimental animals. These mice were preliminarily raised for 1 week with commercial solid feed, and 7 mice were used as one group in the test. MF powder feed blended with 0.5% cholesterol and 1% cholic acid was used as basal feed, and the basal feed was administered into the control group. The test groups were administered the basal feed further containing 0.3% and 1.0% laver proteins obtained in Example 2 and the basal feed further containing 3.0% lyophilized laver powder, respectively. This administration was performed for 28 days, and after the test was finished, blood was collected from the main artery in the abdomen of the animal under anesthesia with ether, and plasma lipid components (total cholesterol, HDL cholesterol, triglyceride) were quantified. The LDL cholesterol level was determined by subtracting the HDL cholesterol level from the total cholesterol level. The results are shown in Table 3.

TABLE 3

| | Total | HDL | LDL | |
|---|---|---|---|---|
| | cholesterol | cholesterol | cholesterol | Triglyceride |
| | (mg/dl) | (mg/dl) | (mg/dl) | (mg/dl) |
| Ordinary food | 120.1 ± 9.7 | 86.5 ± 8.3 | 33.6 ± 5.0 | 56.4 ± 2.9 |
| Cholesterol- | 249.7 ± 8.1 | 65.3 ± 3.9 | 184.4 ± 3.4 | 93.2 ± 7.5 |
| containing | | | | |
| food (control | | | | |
| group) | | | | |
| Cholesterol- | 162.5 ± 3.3 | 80.9 ± 4.5 | 81.6 ± 5.4 | 59.0 ± 1.1 |
| containing food | | | | |
| 0.3% laver | | | | |
| proteins in | | | | |
| Example 2 | | | | |
| Cholesterol- | 142.8 ± 2.9 | 86.7 ± 5.9 | 56.1 ± 5.4 | 39.2 ± 0.4 |
| containing food | | | | |
| 1.0% laver | | | | |
| proteins in | | | | |
| Example 2 | | | | |
| Cholesterol- | 225.5 ± 2.3 | 70.6 ± 6.9 | 154.9 ± 3.4 | 78.2 ± 3.2 |
| containing food | | | | |
| 0.3% lyophilized | | | | |
| laver powder | | | | |

(Mean ± standard error)

As shown in Table 3, the test group administered the feed mixed with the laver proteins was recognized to show a significant reduction in total cholesterol, LDL cholesterol and triglycerides, as compared with the control group. On the other hand, the test group administered the feed mixed with the lyophilized laver powder was recognized to show a slight reduction in total cholesterol etc., as compared with the control group.

### Test Example 4

Protecting Action on Rats Against Ethanol-Induced Hepatic Insufficiency

Seven-week-old male Wistar rats were used as experimental animals. The rats were preliminarily raised for 1 week, and healthy rats were used in the experiment. On the first day of the experiment, the body weight was measured, and the rats were distributed such that the average weights of the respective groups, each containing 6 animals, were almost equal.

MF powder feed containing 1.0% each of cholesterol and cholic acid was used as basal feed, and 30% ethanol water was used as drinking water. The test groups were given the basal feeds further containing 0.3% and 1.0% laver proteins obtained in Examples 2 and 3.0% lyophilized laver powder, respectively. After the animals were raised for 28 days, serum GOT and GPT were measured. The results are shown in Table 4.

TABLE 4

| | GOT | GPT |
|---|---|---|
| | (Karmen Unit) | (Karmen Unit) |
| Ordinary food | 52.61 ± 2.2 | 16.6 ± 0.7 |
| Cholesterol-containing food, 30% | 292.3 ± 20.1 | 163.9 ± 13.4 |
| ethanol (control) | | |
| Cholesterol-containing food, 30% | 102.7 ± 6.3 | 70.8 ± 14.7 |
| ethanol 0.3% laver proteins in | | |
| Example 2 | | |
| Cholesterol-containing food | 72.8 ± 6.8 | 61.5 ± 18.2 |
| 1.0% laver proteins in Example 2 | | |
| Cholesterol-containing food | 232.6 ± 4.5 | 164.5 ± 6.0 |
| 3.0% lyophilized laver powder | | |

(Mean ± standard error)

As shown in Table 4, both the test groups administered the feeds mixed with the laver proteins were recognized to show a significant reduction in the GOT level and GPT level, as compared with the control group. On the other hand, the test group administered the feed mixed with the lyophilized laver powder was recognized to show a slight reduction in the GOT level as compared with the control group, but no change in the GPT level was recognized.

### Test Example 5

Peripheral Blood Vessel-Expanding Action on Rabbits

The laver proteins obtained in Example 1 were dissolved in 10 ml distilled water and orally administered via a cannula into 13-week-old rabbits (New Zealand white species) in doses of 0.3 g/kg, 1 g/kg and 3 g/kg respectively. This oral administration was conducted after blood vessels could be easily observed by removing hair from rabbit ears. Before administration and 10 minutes, 30 minutes, 1 hour and 2 hours after administration, blood vessels were observed and photographed, and the expansibility of blood vessels was calculated with an imaging scanner. As a comparative sample, finely divided dry powder of laver was used in the same manner. The results are shown in Table 5.

TABLE 5

| | | Blood vessel expansibility (%) | | | | |
|---|---|---|---|---|---|---|
| | | | 10 | 30 | | |
| | Admin- | | minutes | minutes | 1 hour | 2 hours |
| | istra- | before | after | after | after | after |
| | tion | admin- | admin- | admin- | admin- | admin- |
| | dose | istra- | istra- | istra- | istra- | istra- |
| | (g/kg) | tion | tion | tion | tion | tion |
| Water-soluble | 0.3 | 100 | 110 | 135 | 133 | 126 |
| laver proteins | 1 | 100 | 105 | 140 | 158 | 152 |
| in Example 1 | 3 | 100 | 118 | 148 | 155 | 172 |
| Finely divided | 1 | 100 | 100 | 98 | 120 | 122 |
| dry powder of | 3 | 100 | 98 | 110 | 130 | 128 |
| laver | 10 | 100 | 101 | 120 | 140 | 144 |

* Blood vessel expansibility is calculated assuming that the value of blood vessels before administration is 100%.

As shown in Table 5, the animals administered the laver proteins were observed to expand blood vessels as compared with those before administration. On the other hand, the animals administered the finely divided dry powder of laver were also observed to expand blood vessels, but this action was lower than by the laver proteins.

### Test Example 6

Blood Viscosity-Reducing Action

The influence of ingestion of the laver proteins obtained in Example 2 on blood viscosity in humans was examined. Each of three volunteers A, B and C was requested to eat much meat on the night before the test and to drink 100 ml water on the next morning, and 1 hour later, 9.5 ml blood was collected in a hypodermic syringe containing 0.5 ml heparin and then stirred, and immediately the passage time of 100 μl whole blood was measured by a cell rheology measuring instrument MC-FANKH-3 (Hitachiharamachi Denshi Kogyo Co., Ltd.). After measurement, 2 g laver proteins obtained in Example 2, together with 100 ml water, were orally ingested by each of the volunteers, and 2 hours later, blood was collected, and the passage time of 100 μl whole blood was measured. The results are shown in Table 6.

TABLE 6
Passage time of 100 μl whole blood (sec)

| | Before eating | | 2 hours after |
|---|---|---|---|
| | meat on the | Next | ingestion of the |
| | previous day | morning | laver proteins |
| A | 32.0 | 37.3 | 32.1 |
| B | 33.2 | ∞ | 33.3 |
| C | 32.3 | 33.8 | 32.3 |

As shown in Table 6, the three volunteers A, B and C showed a prolonged passage time of 100 μl blood after eating meat, but 2 hours after the laver proteins were ingested, the passage time was returned to the measurement level before eating meat on the previous day.

### Example 3

| Soft drink | | |
|---|---|---|
| The laver proteins prepared in Example 1 | 1% | by weight |
| Sugar | 15% | by weight |
| Conc. lemon juice | 1% | by weight |
| Thickened polysaccharides | 0.2% | by weight |
| Yogurt flavor | 0.1% | by weight |
| Water | 82.7% | by weight |

The above components were mixed, bottled and sterilized to produce a laver protein-containing soft drink.

### Example 4

| Health supplementary food | | |
|---|---|---|
| The laver proteins prepared in Example 2 | 80% | by weight |
| Lactose | 19.5% | by weight |
| Sucrose fatty esters | 0.5% | by weight |

The above components were mixed and tabletted into tablets with a tabletting machine, to give a health supplementary food.

In the present invention, physiological activities possessed potentially by seaweeds of the genus Porhyra can be efficiently exhibited to contribute to maintenance and promotion of health as described above.

What is claimed is:

1. A laver protein-containing composition obtained by adding water, a saline solution or an aqueous dilute alkali solution to seaweeds of the genus Porphyra or finely divided dry particles thereof, wet milling the materials to extract soluble components therefrom, and separating proteins form the extract.

2. The laver protein-containing composition according to claim 1, which is obtained by successive extraction with water, a saline solution and an aqueous dilute alkali solution respectively.

3. The laver protein-containing composition according to claim 1, wherein the protein is separated by a protein precipitation method with ethanol.

4. The laver protein-containing composition according to claim 1, wherein the protein is separated by a method using a dialysis membrane.

5. The laver protein-containing composition according to claim 1, wherein the protein is separated by a protein precipitation method using an organic solvent other than ethanol or with ammonium sulfate, an isoelectric precipitation method, or an ion-exchanger adsorption method.

6. Health foods having a blood pressure-dropping action, a hepatic function-improving action, a lipid metabolism-improving action, a peripheral blood vessel-expanding action and a blood viscosity-reducing action, which comprises the laver protein-containing composition of claim 1 as a major component.

7. The foods according to claim 6, wherein the foods are soft drinks.

8. The foods according to claim 6, wherein the foods are in the form of tablets.
